(19) United States
(12) Plant Patent Application Publication
(10) Pub. No.: US 2002/0092083 P1
(43) Pub. Date: Jul. 11, 2002

(54) ZOYSIAGRASS PLANT 'HIMENO'

(76) Inventor: Yoshifumi Miyachi, Hiroshima (JP)

Correspondence Address: OLIFF & BERRIDGE, PLC, P.O. BOX 19928, ALEXANDRIA, VA, 22320, US

(21) Appl. No.: 09/757,653

(22) Filed: Jan. 11, 2001

(51) Int. Cl. A01H 5/00

(52) U.S. Cl. PLT/390

(57) ABSTRACT

A new and distinct variety of Zoysiagrass plant possessing a bluish deep green color, slow vertical growth, and minimal headings.

### BACKGROUND OF THE INVENTION

### Field of Invention

The present invention relates to a new and distinct asexually reproduced variety of perennial Zoysiagrass (Zoysia japonica).

### SUMMARY OF THE INVENTION

This invention relates to a new and distinct variety of Zoysiagrass plant that was discovered by the inventor growing among other cultivated Zoysiagrass varieties in the inventor's lawngrass collection in Hiroshima, Japan. This new variety of perennial turf grass possesses a unique combination of desirable qualities such as slow vertical growth, minimal headings and a bluish deep green color.

For purposes of registration under the “International Convention for the Protection of New Varieties of Plants” (generally known by its French acronym as the UPOV Convention) and noting sections 1612 of the Manual of Patent Examining Procedure, it is proposed that the new variety of Zoysiagrass of the present invention be named “Himeno Zoysiagrass.”

### BRIEF DESCRIPTION OF THE DRAWINGS

FIG. 1 is a color photograph depicting the leaf blades of Himeno, which exhibit a bluish deep green color of 10GY5/6 using the Munsell Color System. The leaf blade is flat, with a round bottom and gradually tapers to an acute point. The first mature leaf is 3.8-4.9 millimeters in width and 3.0-4.5 centimeters long.

FIG. 2 is a color photograph depicting the spikes of Himeno. The spike contains stigmas of yellowish white color of 1 GY9/1 using the Munsell Color System. The glumes are 2.3 millimeters long and are reddish brown color of 7R2.5/6 using the Munsell Color System.

FIG. 3 depicts DNA testing of Himeno and other Zoysiagrass varieties using CMN-B42 Primer.

FIG. 4 depicts DNA testing of Himeno and other Zoysiagrass varieties using CMN-B46 Primer.

### DETAILED DESCRIPTION OF PREFERRED EMBODIMENTS

Himeno was discovered growing in the inventor's Miyachi and Yano Collection in Hiroshima, Japan. Specifically, Himeno was discovered growing in a pot as progeny of one of five strains of Zoysia japonica which the inventor had previously collected from Yaku Island, which is located in the southern portion of Kyushu, Japan.

After collecting the five strains from Yaku Island, the inventor placed each of the five strains of Zoysia japonica in individual pots in the “Miyachi and Yano Collection,” which is a nursery and research facility specializing in varieties of Zoysia japonica. The five pots were numbered and the origin of the Zoysiagrass was noted. Although the five strains of Zoysiagrass retrieved from Yaku Island all are strains of Zoysia japonica, none of them have variety names since they were existing in an uncultivated state and have not been commercialized. The five strains brought back from Yaku Island each exhibited a very large plant height and a deep yellowish green color, while Himeno is much shorter and has a bluish deep green color. Two of the five strains brought back from Yaku Island were unable to survive and deteriorated. Himeno emerged from one of these two deteriorated pots. The three surviving strains were designated as J-29, J-30 and K-1.

The Miyachi and Yano Collection is located in northern Hiroshima, Japan. The collection is owned and maintained by Zoysian Japan Co., Ltd. Research Institute. The Miyachi and Yano Collection contains over 370 strains of Zoysia japonica collected from all across Japan.

Himeno was discovered growing in sandy-loam soil native to Hiroshima in a pot in the Miyachi and Yano Collection. The location of the Miyachi and Yano Collection has an average mean temperature of 11.5° C. and receives an average of 1.46 meters of rainfall annually. Temperature extremes may approach −10° C. and 33° C. The location of the Miyachi and Yano Collection is 580 meters above sea level.

After discovery, the inventor asexually reproduced Himeno by cutting stolons and rhizomes, rooting them in soil and planting the rooted material to provide planting stock for studying performance and for comparison.

This newly discovered variety of Zoysiagrass is a perennial that exhibits an attractive and striking bluish deep green color, slow vertical growth, minimal headings, low temperature hardiness, low vertical growth, stable growth, and durable stolon. The most distinguishable characteristics of Himeno are its bluish deep green color, slow vertical growth, and minimal headings.

Himeno is a durable turf grass well suited to recreation areas and requires less maintenance in comparison to other popular Zoysiagrass varieties such as Meyer and Emerald. The leaf is very short and dense. The stolon is relatively thick and very tough compared to other Zoysiagrass. The grass exhibits very good tolerance to wear and consolidation. It is also quick spreading and has minimal water and fertilizer requirements. The low plant height of Himeno reduces mowing frequency. Thus, Himeno is well adapted to use in sports fields, parks and for water and wind erosion control.

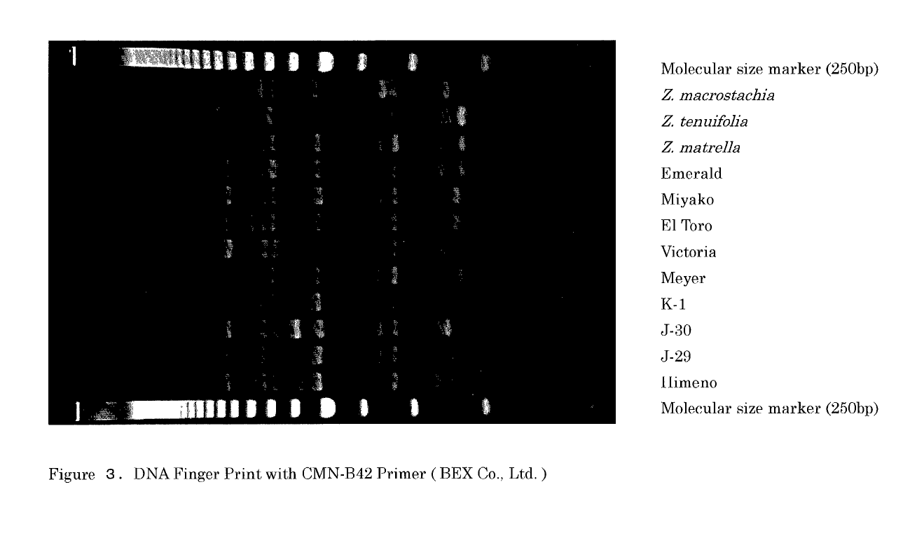

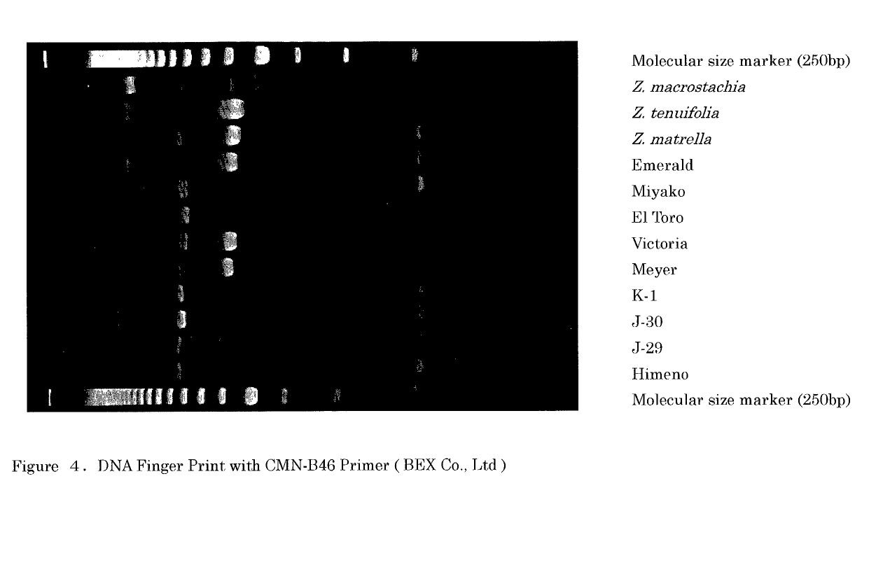

FIG. 3 depicts DNA testing of Himeno and other Zoysia varieties using the RAPD method and CMN-B42 Primer. FIG. 4 depicts DNA testing of Himeno and other Zoysia species using the RAPD method and CMN-B46 Primer. In each testing, template DNA was extracted from the mature leaves of Himeno, J-29, J-30, K-1, Meyer, Victoria, El Toro, Miyako, Emerald, Zoysia matrella, Zoysia tenuifolia, and Zoysia macrostachia and was replicated with primer using PCR techniques. The amplified products were electorophoresised on agarose gel, stained and photographed. As can be ascertained from the DNA testing, Himeno is distinguishable from the three strains retrieved from Yaku Island (J-29, J-30 and K-1) and the other above mentioned Zoysiagrass varieties.

A detailed description of the Himeno Zoysiagrass:

(a) The leaves of Himeno exhibit a bluish deep green color of 10GY5/6 using the Munsell Color System.

(b) The grass grows fast horizontally with an erect very low-growth characteristic.

(c) The grass spreads by stolons and rhizomes, forming the highest dense upright growth among the known varieties of Zoysia japonica with a uniform surface and with an extensive root system. The stolons are a very dark red color, 1R2.5/2 using the Munsell Color System. The inteniodes are 2.0-3.0 centimeters apart and have a diameter of 1.5-2.0 millimeters.

(d) The headings are minimal and the culms are very short. The culms do not rise above plant height.

(e) The leaves are rolled in a bud shoot.

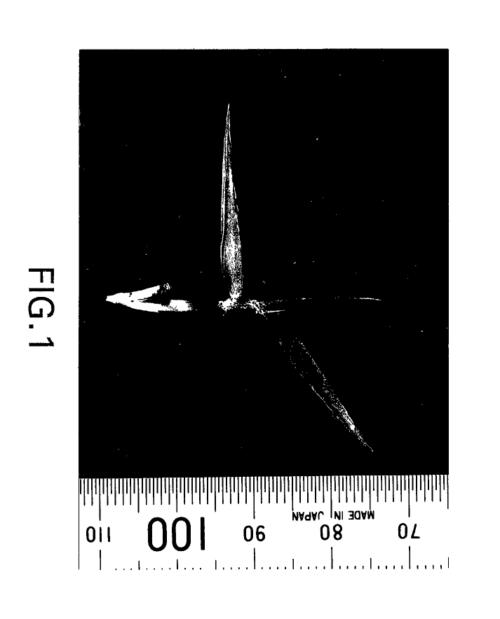

(f) The leave blade is flat, with a round bottom, and gradually tapers to an acute point (see FIG. 1).

(g) The first mature leaf is 3.8-4.9 millimeters in width and 3.0-4.5 centimeters long.

(h) The leaf blade has a few hairs.

(i) The ligule is a fringe of hairs.

(j) Auricles are absent.

(k) The collar is broad and continuous.

(l) The sheath is glabrous, and split with separate margins.

(m) The entire plant is glabrous, except on the upside of the leaf blade and on the inside of the collar just above the ligule, where several long hairs are present.

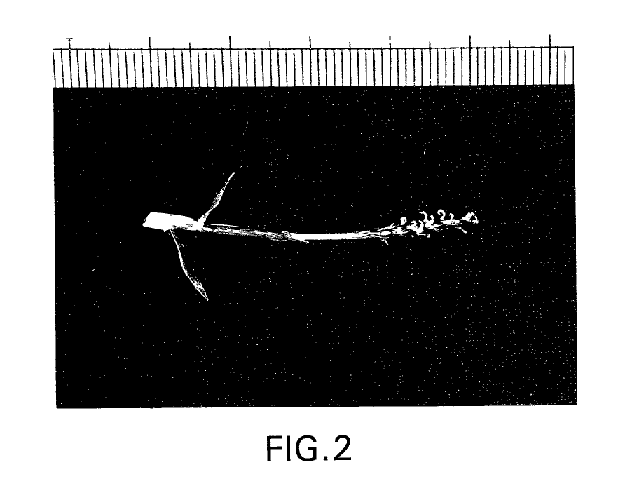

(n) The inflorescence consists of a single spike at the top of the main stem (see FIG. 2).

(o) The spikelet contains stigmas of yellowish white color of 1 GY9/1 using the Munsell Color System. The glumes are 2.3 millimeters long and are reddish brown color of 7R2.5/6 using the Munsell Color System.

The Meyer variety is the closest known variety of Zoysiagrass to Himeno. A description of Meyer is:

(a) Meyer exhibits a deep green leaf color of 7GY5/6 using the Munsell Color System.

(b) Meyer is slightly low growing horizontally, with an erect low growth characteristic.

(c) Meyer spreads by stolons and rhizomes, forming a high dense upright, uniform surface with an extensive root system. The stolons are deep red color of 1R2.5/6 using the Munsell Color System. Meyer has intemodes 3.2-4.2 centimeters apart and 1.3-1.8 millimeters in diameter.

(d) The heading is marked.

(e) The leaves are rolled in a bud shoot.

(f) The leaf blade is flat, with a round bottom and gradually tapers to an acute point.

(g) The first mature leaf is 3.8-4.9 millimeters in width and 3.0-4.5 centimeters long.

(h) The leaf blade has a few hairs.

(i) The ligule is a fringe of hairs.

(j) Auricles are absent.

(k) The collar is broad and continuous.

(l) The sheath is glabrous and split with separate margins.

(m) The entire plant is glabrous, except on the upside of the leaf blade and on the inside of the collar just above the ligule, where several long hairs are present.

(n) The inflorescence consists of a single spike at the top of main stem.

(o) The spikelet contains stigmas of pale pink color of 7R9/1 using the Munsell Color System. The glumes are 2.3 millimeters long and are a dark red color of 4R2.5/6 using the Munsell Color System.

What is claimed is:

1. A new and distinct variety of Zoysiagrass plant as described and illustrated herein.
